(19) United States
(12) Patent Application Publication
(10) Pub. No.: US 2020/0341756 A1
(43) Pub. Date: Oct. 29, 2020

(54) Agile human resources method within the pharmacoinformatics field

(71) Applicant: Susan M. Stellflue, Lake Zurich, IL (US)

(72) Inventor: Susan M. Stellflue, Lake Zurich, IL (US)

(21) Appl. No.: 16/914,713

(22) Filed: Jun. 29, 2020

Related U.S. Application Data
(60) Provisional application No. 62/995,098, filed on Jan. 13, 2020.

(51) Int. Cl. G06F 8/77; G06Q 10/10; G06Q 10/06; G16H 40/20; G16H 10/20; G16H 70/40; G06Q 30/00

(52) U.S. Cl. CPC G06F 8/77; G06Q 10/103; G06Q 10/067; G06Q 10/063118; G06Q 30/018; G16H 40/20; G16H 10/20; G16H 70/40; G06Q 10/1053; G06Q 10/101

(57) ABSTRACT

Described within is an agile human resources (HR), or agile HR, method for modeling agile development team structure within the pharmacoinformatics field of study. As agile adoption continues to span outside the traditional sphere of agile software development, to cover an evolving set of cross-functional informatics and quantitative specialties, there is a need for a defined agile human resources process. The steps to this process include: receiving requirements for an agile technology project from one or more customers; identifying any project stakeholders; identifying said project is primarily within a pharmacoinformatics field of study; identifying that an agile development team is to be modeled; identifying at least one agile development methodology to utilize in modeling; generating a team structure model document; reviewing with any project stakeholders and talent acquisition providers; and managing any agile team structure changes.

### REFERENCE TO RELATED APPLICATION

The present application claims priority to the earlier filed provisional application having Ser. No. 62/995,098, filed on Jan. 13, 2020, and hereby incorporates subject matter of the provisional application in its entirety.

### SUMMARY OF THE INVENTION

The present subject matter relates to agile human resources, or agile hr, and more specifically, to methods of modeling and management of agile team structure for science and technology projects within the field of pharmacoinformatics.

### BRIEF DESCRIPTION OF INVENTION DRAWINGS

The applicant has attached the following illustrations of the aforementioned invention at the end of this provisional patent application:

FIG. 1: a flow chart drawing providing a visual representation of the method claims.

FIG. 2: a Venn-diagram depicting different knowledgebase specialties grouped into a ten-person agile team structure working on one technology product.

### DETAILED DESCRIPTION

The present disclosure provides an embodiment of a method comprising of agile human resources (HR) management, or agile HR, to model candidate team structure for agile technology development within the field of pharmacoinformatics. The embodiments of this method comprise of: receiving requirements for an agile technology project from one or more customers; identifying any project stakeholders; identifying said project is primarily within a pharmacoinformatics field of study; identifying that an agile development team is to be modeled; identifying at least one agile development methodology to utilize in modeling; generating a team structure model document; reviewing with any project stakeholders; collaborating with talent acquisition providers; and managing any agile team structure changes throughout the implementation of the project.

The embodiments include modeling a conventional agile team structure typically with 4 to 10 team members, having one or more agile executive leaders, agile development leaders, and customers. Typically to perform at least one of software, hardware, firmware, digital and quantitative data science development. As agile adoption continues to span outside the traditional sphere of agile software development, to cover an evolving set of cross-functional informatics and engineering specialties, there is a need to define how these cross-functional teams are structured.

The disclosure further adds pharmacoinformatics specialists to collaboratively develop cross-functional technologies.

The embodiments enable structuring of teams from many similar related and known information technology or pharmacy related development technologies and specializations comprising of: medical, bio, cancer (chemo, radiation), molecular, ontology, oncology, toxicology, genome, nuclear, neurology, immunology, metabolic, pharmacy, chemical, magnetic or radiation scanning, pharmacovigilance, biowarfare, counter intelligence, quality, regulatory, compliance, agriculture, horticulture, herbal, veterinary, cosmetic science, packaging science, environmental, recycling, military, materials, robotics, augmented reality, virtual reality, user experience, user research, digital experience, automation, and cybersecurity technologies

The foregoing scientific specialties listed provides an example of various pharmacoinformatics related studies, but is not intended to be exhaustive or to limit the scope of specialties that could potentially be utilized in current or future state.

An implementation may utilize any agile methodology, as an example, the description used within refers to the methodology of extreme programming XP, and its subset of extreme machine learning XL. An agile software development method that was originally introduced in 1996, and thus is a widely utilized agile team structuring model. The iterative and collaborative methodology may be applied to other development settings as well. The XP agile human resources process applies the concepts of co-located teams and paired programming, where two or more engineers work on one computer, in one room at the same time, on one section of technology code or hardware.

In an implementation, the known agile methodology of extreme machine learning XL may also be utilized. This is a further subset of extreme programming, and a known quantitative development methodology and collaborative team modeling process that was originally introduced circa 2006. This methodology further extends concepts from extreme programming XP by adding in quantitative data scientists who design algorithms for machine learning in conjunction with extreme programming engineers. Alternatively, placing two or more quantitative data scientists in the team structure model developing quantitative applications instead of software engineers. Extreme machine learning frequently results in a triad or quad of quantitative analysts and software engineers developing technology solutions collaboratively.

As exampled, by joining team members who are trained in cooperative methodology known as paired, triad, or mob development in computer programming or quantitative technologies alongside: (a) those trained in drug-related sciences and compliance with (b) those trained in various automation processes, industry will gain advancement in drug-related informatic science that creates more reliable and compliant technologies.

In an implementation wherein identifying the aforementioned key metrics of the agile team structure and methodologies to be used; at what iterations or stages of development; an agile team structure model document is drafted. Further, collaborating with talent acquisition providers to facilitate any hiring, both internal or external, needed to fully form cross-functional teams. Further collaborating with at least one of agile executive leaders, agile development leaders, customers, or any other key project stakeholders wherein managing any changes or re-training throughout the project. As agile human resources embraces and expects change throughout the iterations of an associated agile project.

The government, academia, and business demographic entities that would use this invention include:
a) Pharmaceutical, biotechnology, healthcare, veterinary, and dental industry entities for research and for accurate patient drug-related treatments. Potentially improving pharmacy dosing accuracy. Additionally, to avoid pharmaceutical and chemical pollution to humans and the living ecosystem caused by over-prescribing or over-estimating drug dosages.
b) Government entities or military for biowarfare, nuclear, or chemical counter intelligence to avoid damage or pollution to the environment, humans, and the ecosystem in the unfortunate instance of disaster or warfare. The embodiments would be particularly useful in times of disaster or during wartime efforts to minimize impact on living species and the climate in the event of a chemical or toxicology disaster.
c) Industry, academia research, or government entities in the artificial intelligence or high-tech field of study to create a variety of pharmacoinformatics or pharmacovigilance related technology products.

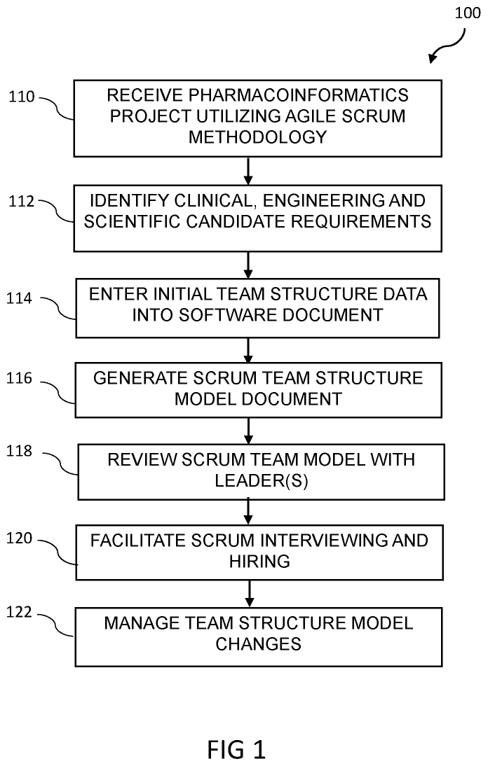

As an example, FIG. 1 is a flow chart drawing providing a visual representation demonstrating the process to create an agile team structure model 100. In this example, the initial project requirements have been established, and the product variables include: a) a product that is a pharmacoinformatics product; b) the project is to be in scrum methodology. The steps to establish the agile scrum team structure model are depicted. The elements include: receive pharmacoinformatics project utilizing agile scrum methodology 110; identify clinical, engineering and scientific candidate requirements 112; enter initial team structure date into software document 114; generate scrum team structure model document 116; review scrum team model with leaders 118; facilitate scrum interviewing and hiring 120; and manage scrum team structure model changes 122.

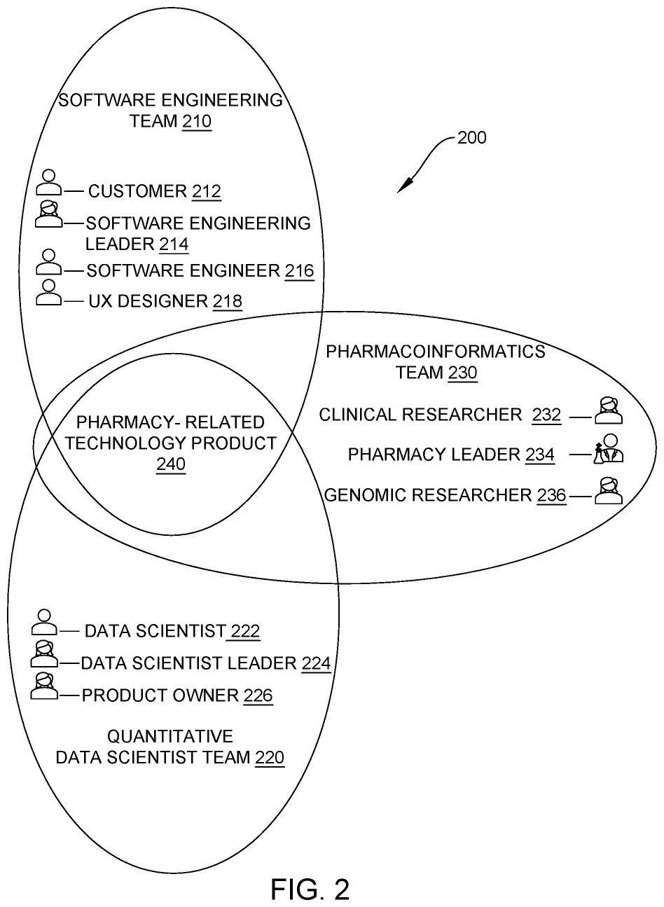

As an example, FIG. 2 visualizes a complete ten-person agile team structure 200. A model where the team is practicing the methodologies of extreme programming (XP) and extreme machine learning (XL). Embodiments include three co-located uniquely technical teams. As detailed, there is a core agile software engineering team 210; quantitative data science team 220; further joined by a pharmacoinformatics team 230 together working on the end pharmacy-related technology product 240. Each grouping having a leader. This models a typical team structure that may be present in agile cross-functional development project. The embodiments work together and collaborate, however each grouping or an individual team member may not be present for the entirety of product development.

Table of agile team members found in FIG. 2:

| | |
|---|---|
| 212 | Customer |
| 214 | Software Engineering Leader |
| 216 | Software Engineer |
| 218 | UX Designer |
| 222 | Data Scientist |
| 224 | Data Scientist Leader |
| 226 | Product Owner |
| 232 | Clinical Researcher |
| 234 | Pharmacy Leader |
| 236 | Genomic Researcher |

It is important to recognize that it is impractical to describe every conceivable combination of subject innovations, or embodiments for purposes of describing the claimed subject matter either in current state or future state. However, a person having ordinary skill will recognize that many further combinations and permutations of the subject innovations are possible. Accordingly, the claimed subject matter is intended to cover all such alterations, modifications and variations that are within the spirit and scope of the claimed subject matter. Any benefits, advantages, or solutions to problems that are described herein with regard to specific embodiments are not intended to be construed as a critical, required, or essential feature or element of any or all of the claimed subject matter.

What is claimed is:

1. A method of agile human resources management to model candidate team structure comprising of:
receiving requirements for an agile technology project from one or more customers;
identifying any project stakeholders;
identifying said project is primarily within a pharmacoinformatics field of study;
identifying that an agile development team is to be modeled;
identifying at least one agile development methodology to utilize in modeling;
generating a team structure model document;
reviewing with any project stakeholders;
collaborating with talent acquisition providers;
and managing any agile team structure changes throughout the implementation of the project.

2. The method of claim 1, further comprising of: receiving approved agile or hybrid-agile development project requirements; comprising of at least one of a software, firmware, hardware, digital, or quantitative technology products.

3. The method of claim 1, further comprising of: identifying one or more agile executives; wherein an approved project may be comprised of multiple agile teams working on similarly related products under the directive of one or more agile executive leaders; referred forward as agile executive leaders.

4. The method of claim 1, further comprising of: identifying one or more agile leadership team members; referred forward as agile development leaders.

5. The method of claim 1, further comprising of: identifying said development project is primarily a pharmacoinformatics project, a field of study covering bioinformatics, immunoinformatics, toxicoinformatics, chemoinformatics, nuclear informatics, radiation informatics, healthinformatics, neuroinformatics, genoinformatics, metabolomoinformatics, pharmacovigilance, and drug-related development within the field of informatics; which may contain one or more products.

6. The method of claim 5, further comprising of: identifying that an agile development team is to be structured for said development project.

7. The method of claim 6, further comprising of: identifying at least one agile methodology to utilize in modeling team structure for said development project.

8. The method of claim 7, further comprising of: modeling from agile team modeling principles wherein typical agile team models comprise of 4 to 10 team members which may include a coach, a product owner, a user experience designer, a quality tester, two or more of software engineers or quantitative data scientists, and any candidate specialists needed based on the requirements of said development project.

9. The method of claim 8, further comprising of: receiving from at least one customer, agile executive leader or agile development leader, any available data identifying: one or more scientific, engineering, or specialist candidates that would be utilized; at what stage or iteration of development said candidates would be utilized; an anticipated number of agile team members needed; any existing employees or contractors that could be used; any re-training that could be done; any new candidate hiring that is expected; any consultant hiring that is expected; geographic work locations; timeline data; licenses and certification requirements; access authorization or security clearance requirements; USCIS immigration constraints; salary or fees budgetary constraints; and any inclusive candidate demographics desired.

10. The method of claim 9, further comprising of: using a computer and software to input said data into a software document.

11. The method of claim 10, further comprising of: generating the first itineration of a team structure model document.

12. The method of claim 11, further comprising of: communicating with said project stakeholders; customers; agile executive leaders; agile development leaders to review said team structure model document, and making any modifications found necessary.

13. The method of claim 12, further comprising of: implementing said agile team structure model by advising with said talent acquisition providers to communicate the team structure model document;
relaying any gathered information for the writing of job descriptions, talent sourcing, interviewing, and hiring of talent;
reviewing any pre-vetted candidates from the talent acquisition providers;
reviewing candidates with one or more project stakeholders, customers, agile executive leaders or agile development leaders;
facilitating agile interviews, facilitating hiring, and making appropriate changes to the team structure model document as needed.

14. The method of claim 13, further comprising of: managing any changes to the agile team structure as work progresses through the iterations.
